(19) United States
(12) Patent Application Publication
(10) Pub. No.: US 2025/0375378 A1
(43) Pub. Date: Dec. 11, 2025

(54) mRNA LIPID NANOPARTICLES FOR THE TREATMENT OF NIEMANN-PICK TYPE C DISEASE

(71) Applicants: Mark Schultz, Iowa City, IA (US); Anna Schwendeman, Dearborn, MI (US); Troy Halseth, Dearborn, MI (US)

(72) Inventors: Mark Schultz, Iowa City, IA (US); Anna Schwendeman, Dearborn, MI (US); Troy Halseth, Dearborn, MI (US)

(21) Appl. No.: 19/229,666

(22) Filed: Jun. 5, 2025

Related U.S. Application Data
(60) Provisional application No. 63/656,343, filed on Jun. 5, 2024.

(51) Int. Cl. A61K 9/1271; A61K 9/51; A61K 31/7105; A61K 38/17; A61P 3/00; B82Y 5/00

(52) U.S. Cl. CPC A61K 9/1271; A61K 9/5123; A61K 9/5192; A61K 31/7105; A61K 38/1709; A61P 3/00; B82Y 5/00

(57) ABSTRACT

Composition and methods for treating Niemann-Pick disease type C are disclosed, utilizing lipid nanoparticles (LNPs) encapsulating mRNA sequences coding for NPC1 and/or NPC2 proteins.

### PRIORITY

This application claims the benefit of the filing date of U.S. Application No. 63/656,343, filed on Jun. 5, 2024, the disclosure of which is incorporated by reference herein.

### INCORPORATION BY REFERENCE OF SEQUENCE LISTING

This application contains a Sequence Listing which has been submitted electronically in ST26 format and hereby incorporated by reference in its entirety. Said ST26 file, created on Jun. 4, 2025, is named 875247US1.xml and is 11,364 bytes in size.

### BACKGROUND

Niemann-Pick disease type C (NPC) is a rare progressive genetic disorder characterized by an inability of the body to transport cholesterol and other fatty substances (lipids) inside of cells. This leads to the abnormal accumulation of these substances within various tissues of the body, including brain and liver tissue. The accumulation of these substances damages the affected areas. NPC is highly variable and the age of onset and specific symptoms can vary from one person to another, sometimes even among members of the same family. NPC can range from a fatal disorder within the first few months after birth (neonatal period) to a late onset, chronic progressive disorder that remains undiagnosed well into adulthood. Most cases are detected during childhood and progress to cause life-threatening complications by the second or third decade of life. NPC is caused by mutations in the NPC1 gene (NPC type 1C) or the NPC2 gene (NPC type 2C) and is inherited in an autosomal recessive manner.

### SUMMARY

Provided herein is a RNA therapeutic for Niemann-Pick C disease. In one embodiment, RNA coding for NPC1 and/or NPC2 is administered to a subject in need thereof. In one embodiment, the RNA is in a lipid particle. After administration, the RNA is translated and NPC1 or NPC2 protein synthesis occurs to provide functional NPC1 and/or NPC2 protein to said subject.

In one embodiment, the disclosure provides a composition comprising lipid nanoparticles encapsulating an mRNA sequence coding for NPC1 protein and/or NPC2 protein. The lipid nanoparticles comprise a combination of SM-102, DMG-PEG 2000, DSPC, and cholesterol. In some embodiments, the mRNA is codon optimized and may include one or more modifications such as N1-methylpseudouridine, a 5′ cap, or a polyA tail. Additionally, the NPC1 protein is encoded by a nucleic acid corresponding to SEQ ID NO: 2 or having at least 80% identity thereto, while in other embodiments the NPC2 protein is encoded by a nucleic acid corresponding to SEQ ID NO: 4 or having at least 80% identity thereto. In further embodiments, the lipids are present in a molar ratio of 50:38.5:10:1.5, and the composition may additionally include a carrier such as water, saline, or phosphate-buffered saline. The lipid nanoparticles may be prepared by microfluidic mixing and exhibit an average particle diameter of about 80 to 150 nanometers.

In another embodiment, the disclosure provides a method for treating Niemann-Pick disease, type C1 by administering the composition described above to a subject in need thereof. In some embodiments, the mRNA is codon optimized and may include one or more modifications selected from N1-methylpseudouridine, a 5′ cap, or a polyA tail, with coding of NPC1 via a nucleic acid corresponding to SEQ ID NO: 2 or having at least 80% identity thereto or coding of NPC2 via a nucleic acid corresponding to SEQ ID NO: 4 or having at least 80% identity thereto. Additionally, in some embodiments the lipid components are present in a ratio of 50:38.5:10:1.5, and the method may include the further administration of a carrier. The treatment in certain embodiments results in a decrease of one or more symptoms associated with Niemann-Pick disease, type C1—including cerebellar ataxia, dysarthria, dysphagia, tremor, epilepsy (both partial and generalized), vertical supranuclear palsy, sleep inversion, gelastic cataplexy, dystonia, inturning of one foot when walking, spasticity, hypotonia, ptosis, microcephaly, psychosis, dementia, hearing loss, bipolar disorder, and depressive conditions (which may include hallucinations, delusions, mutism, stupor, or altered liver function)—or an increased level of NPC1 protein.

Various other aspects and embodiments are disclosed in the detailed description and claims that follows.

### BRIEF DESCRIPTION OF THE DRAWINGS

FIG. 1. Overview of RNA therapeutic for Niemann-Pick C disease.

FIG. 2. Lipid particles (LPs) for delivery of mRNA. The lipid particle has low toxicity (1 mg/kg) and shows no insertional mutagenesis. The particle is based off the lipid particle used in the Moderna Covid-2 vaccine and contains the following lipids: SM-102, DMG-PEG 2 k, DSPC and cholesterol.

FIG. 3. Murine NPC1 mRNA from TriLink.

FIG. 4. LP preparation using microfluidic mixing.

FIG. 5. High encapsulation efficiency and homogenous particle production via DLS and scanning microscopy.

FIG. 6. Niemann-Pick C mRNA/protein production in cells.

FIG. 7. LP-GFP mRNA results in high transfection efficiency in several cell types.

FIG. 8. Transfection efficiency is near 100% for many cell types.

FIG. 9. LP-NPC1 caused an increase in total NPC1 protein production.

FIG. 10. LP-NPC1 RNA is EndoR evidencing proper folding and trafficking.

FIG. 11. NPC1 mRNA LNPs corrected cholesterol accumulation.

FIG. 12. Codon-optimized mRNA to improve translation efficiency and mRNA stability (Jorgensen et al. 2005; Hanson and Coller, 2018).

FIG. 13. Codon optimization (co-NPC1) improves expression.

FIG. 14. LP-NPC1 mRNA clears cholesterol and autophagy defects.

FIG. 15. Overview of mouse experiments.

FIG. 16. GFP;LP mRNA injections—48 hours.

FIG. 17. LP produces NPC1.

FIG. 18. Corpus Callosum injections.

FIG. 19. Luciferase assay showing that the particle formulation targets the liver.

FIGS. 20A-20D. Lipid particle-mediated delivery of Npc1 mRNA to Npc1−/− mice increases NPC1 protein and corrects secondary phenotypes. A, B, C, D 3-week-old Npc1+/+ (WT) and Npc1−/− mice were injected once IP with saline (veh), or with 1 mg/kg LP containing GFP mRNA (GFP:LP), or LP containing codon optimized Npc1 mRNA (Co-Npc1:LP) and livers were collected 48 hours later. Liver lysates were analyzed for A total NPC1 protein (quantified at right), B EndoH sensitivity, C LC3B-II (quantified at right) via western blotting or D Hmgcs1 mRNA by qPCR. *p≤0.05, **p≤0.01, ***p≤0.001, ****p≤0.0001 by one-way ANOVA with Tukey's post-hoc test; A F (3, 16)=145.7, C F (3, 12)=30.78, D F (3, 15)=24.09. Data are shown as the mean±SEM from D n=4-5/group, C n=3/group.

FIG. 21. Codon optimized LP restores cholesterol esterification in Npc1−/− mice. 3-week-old Npc1+/+ (WT) and Npc1−/− mice treated with saline (veh), GFP mRNA (GFP:LP), or codon optimized Npc1 mRNA (Co-Npc1:LP). Livers were collected 48 hours after treatment. Levels of cholesterol esters 18:0, 18:1, and 16:0, and 20:3 were analyzed by liquid-chromatography-mass spectrometry analysis. Data are shown as the mean±SEM from n=4-5/group. *p≤0.05, **p≤0.01, ***p≤0.001, ****p≤0.0001 by one-way ANOVA with Tukey's post-hoc test; F (3,15)=19.97, F (3, 15)=35.09, F (3, 15)=26.25, F (3, 15)=16.11, from left to right, top to bottom.

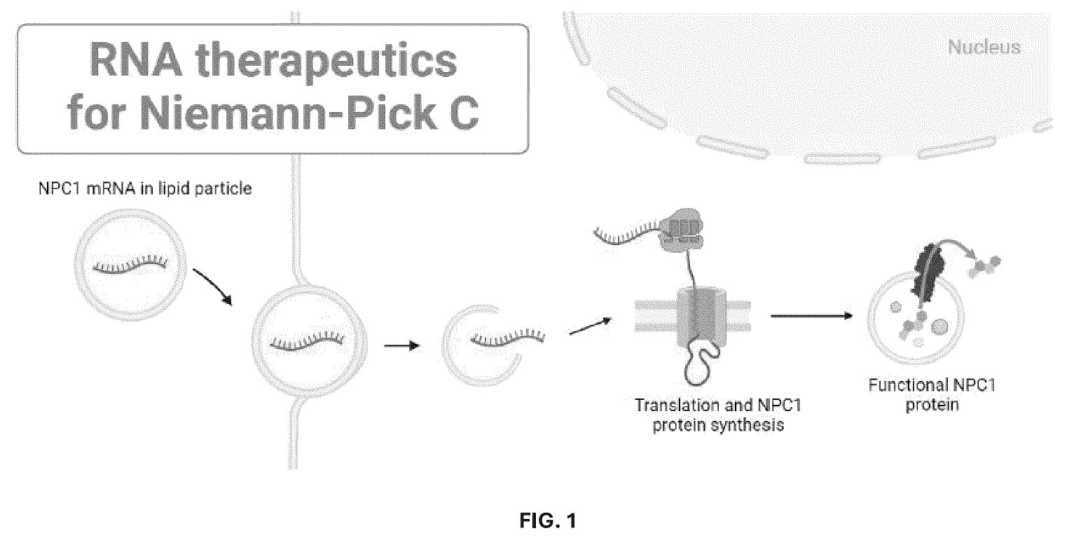

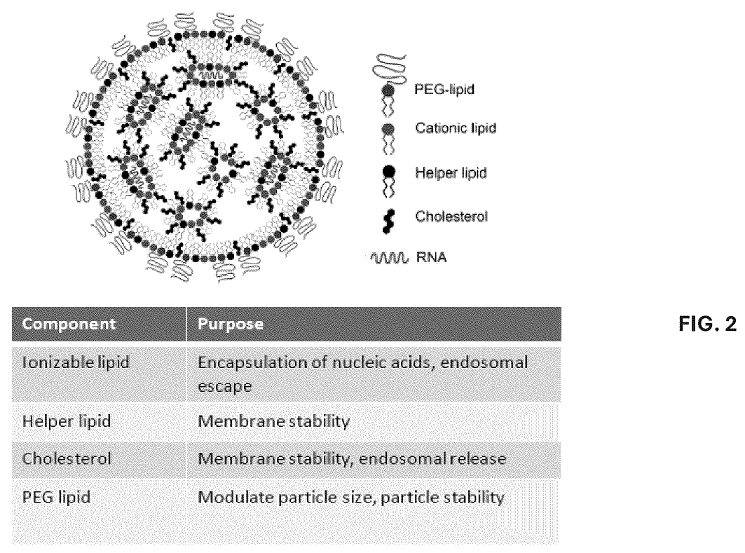

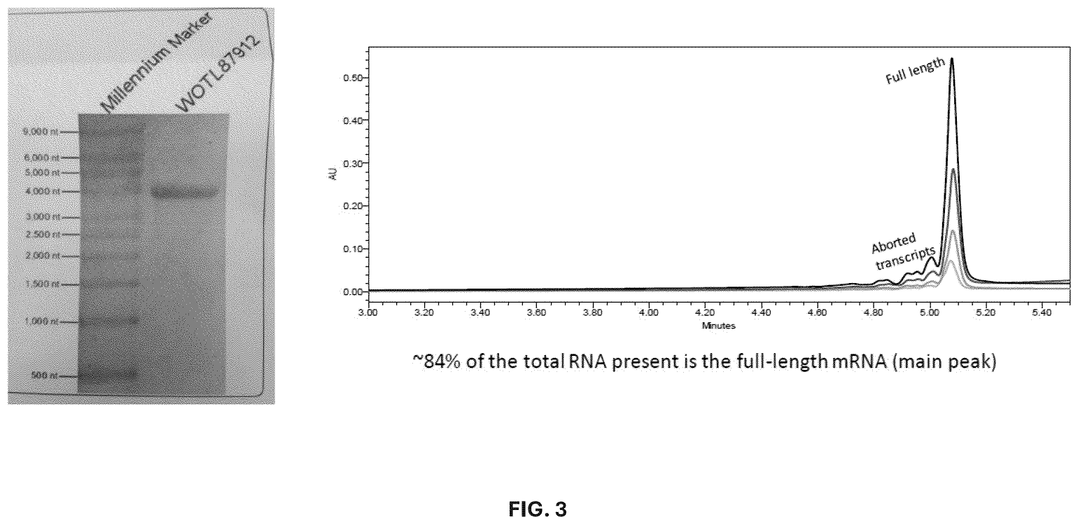

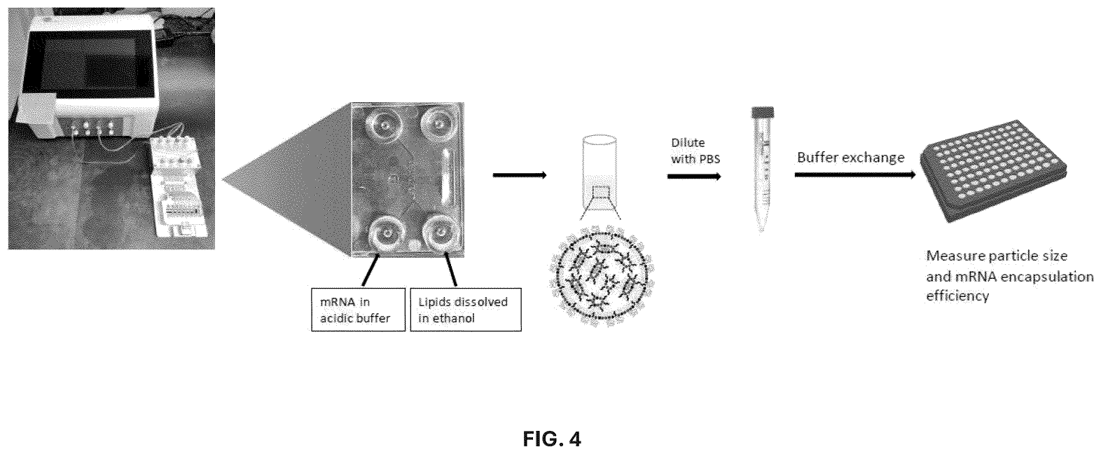

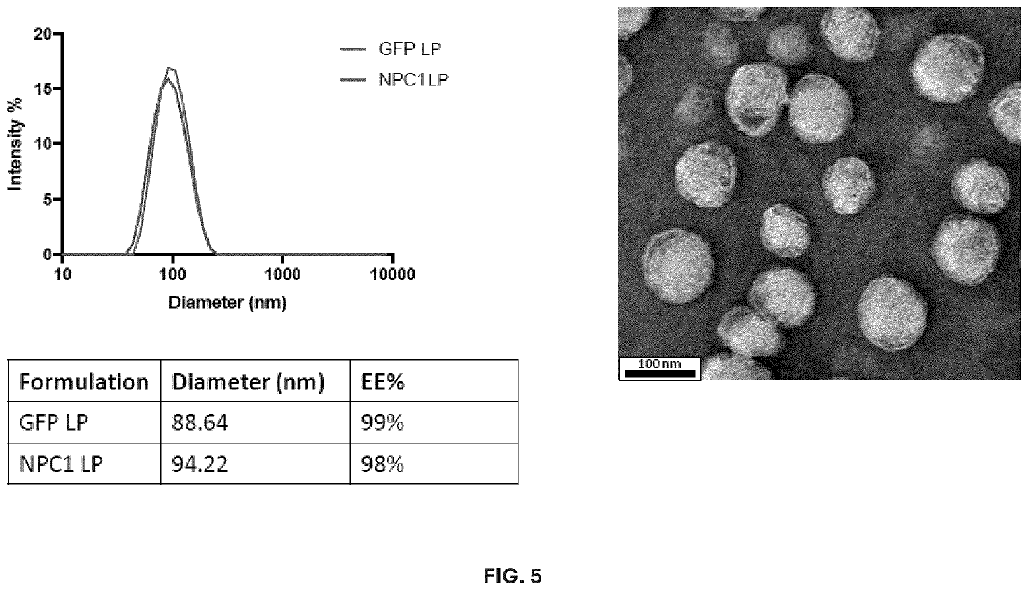

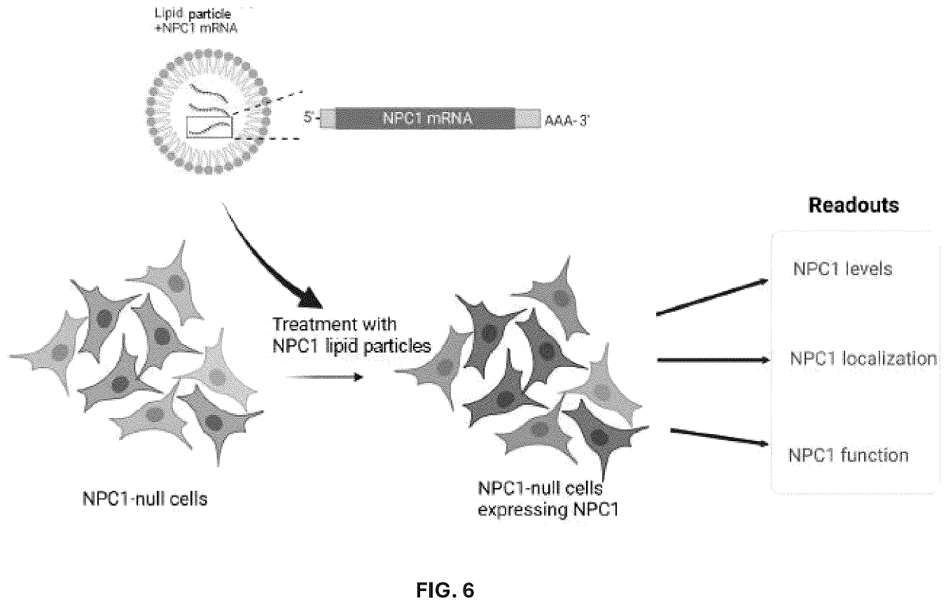

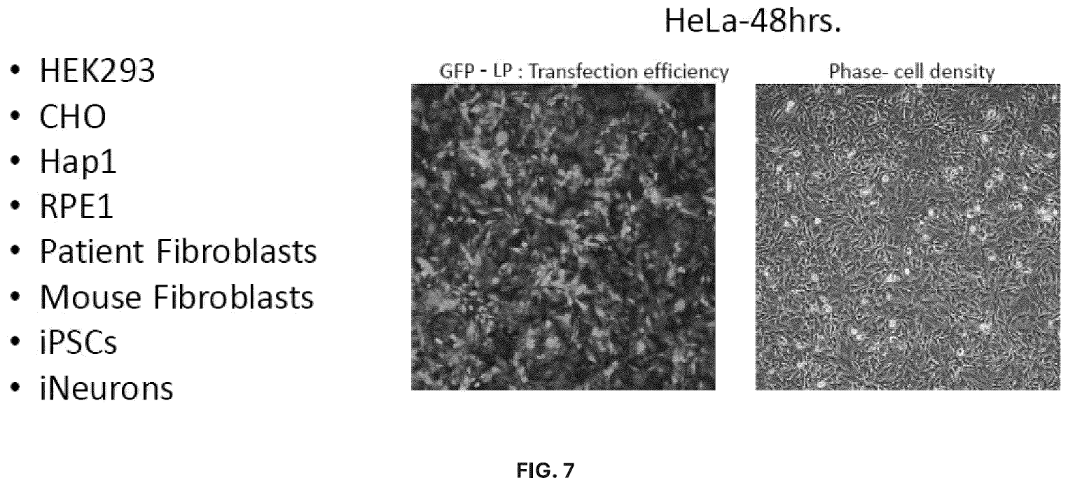

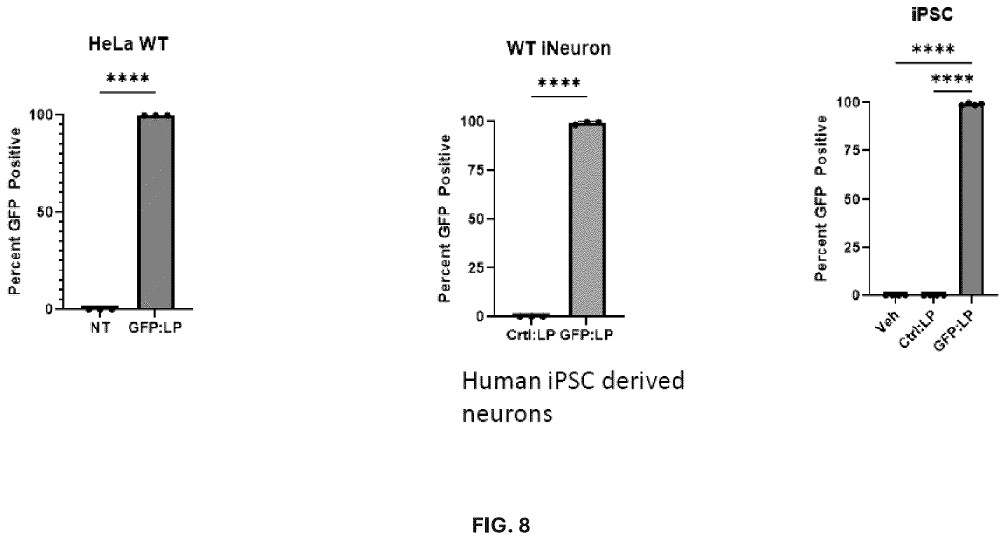

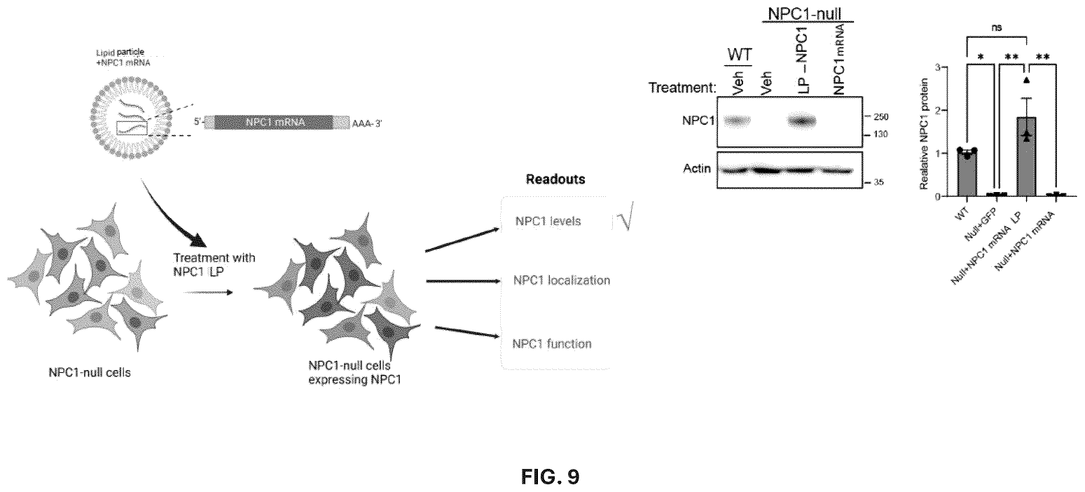

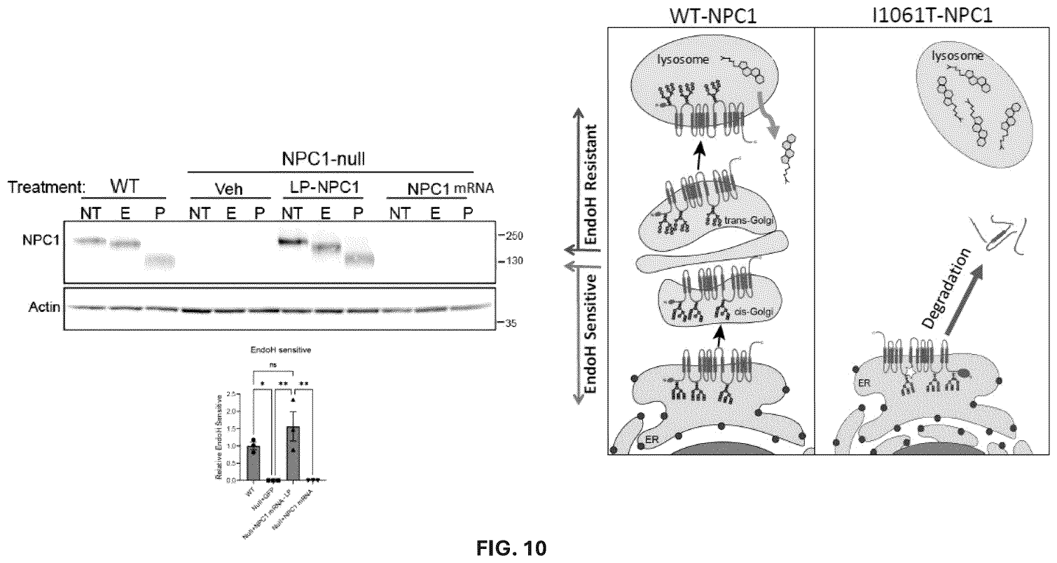

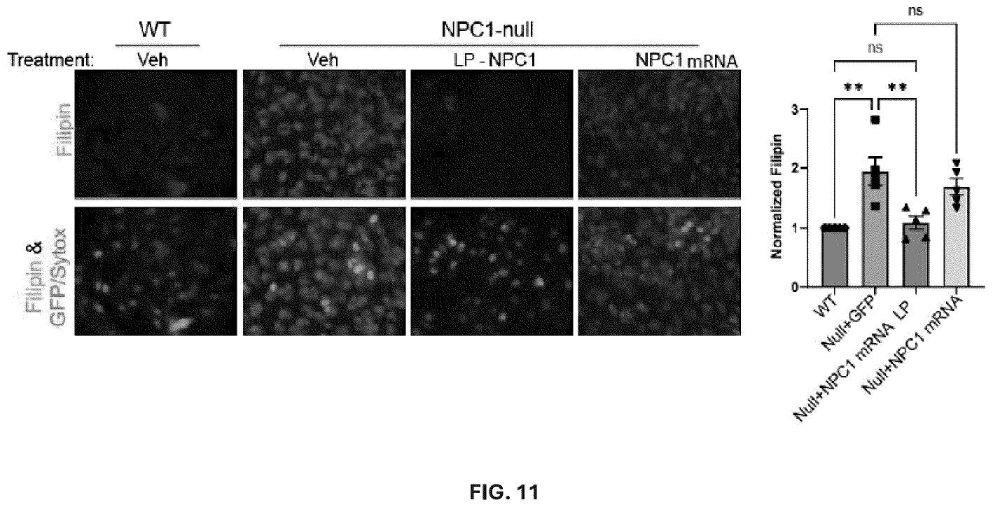

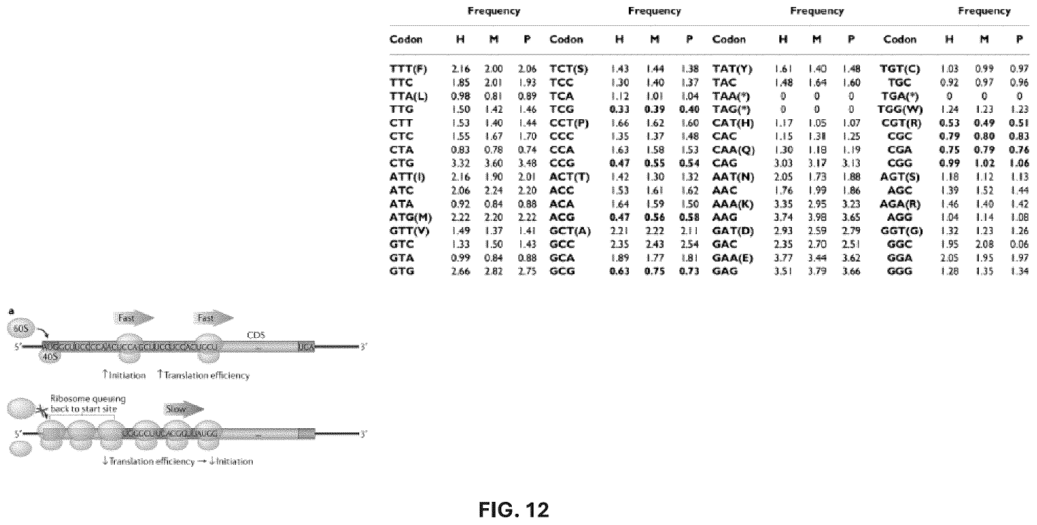

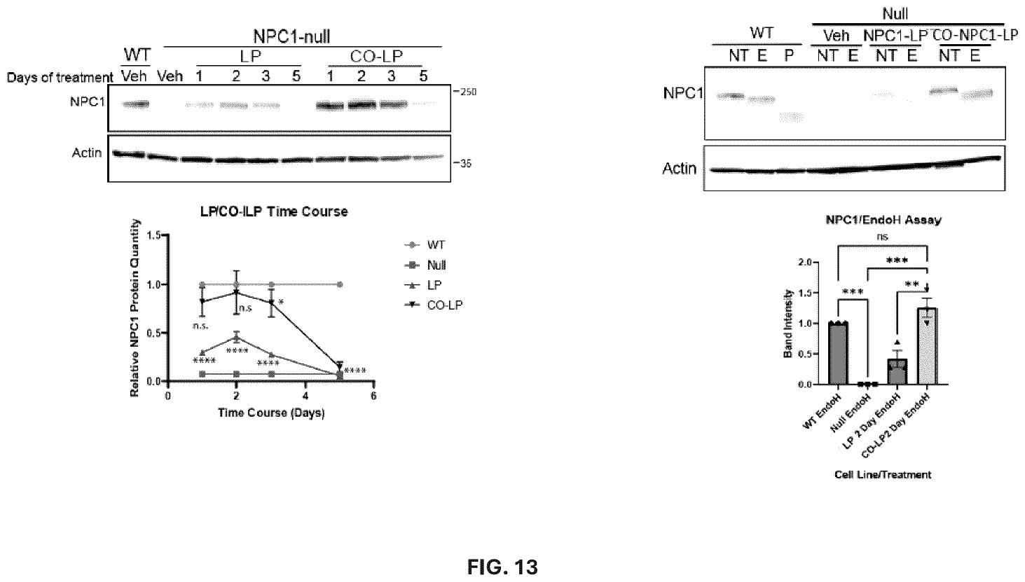

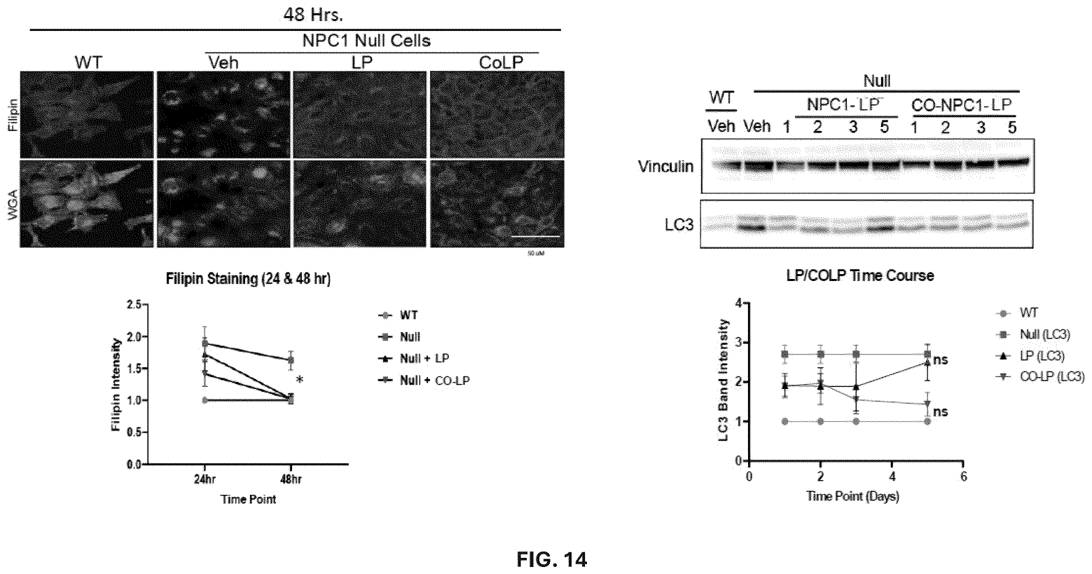

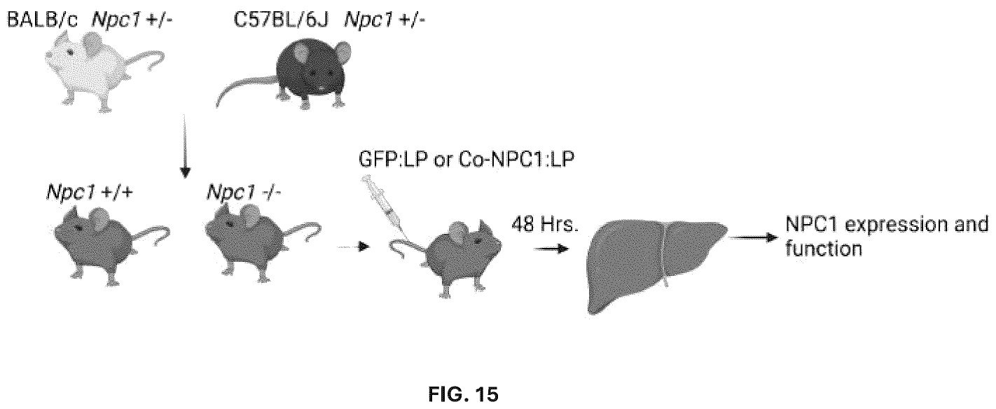

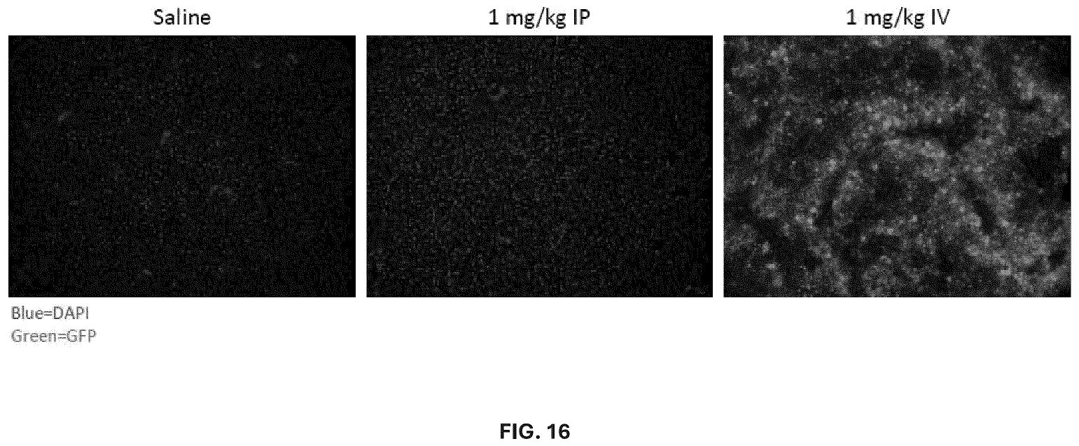

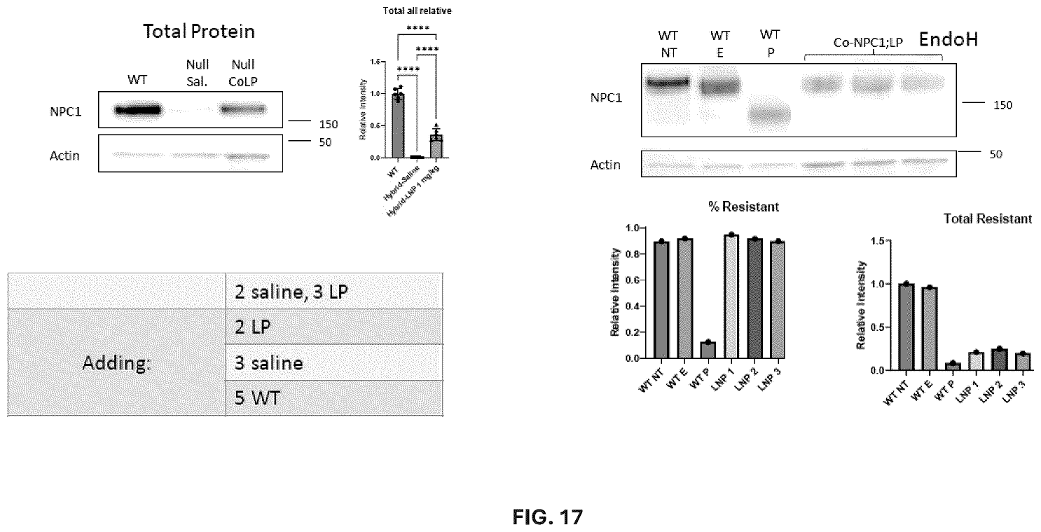

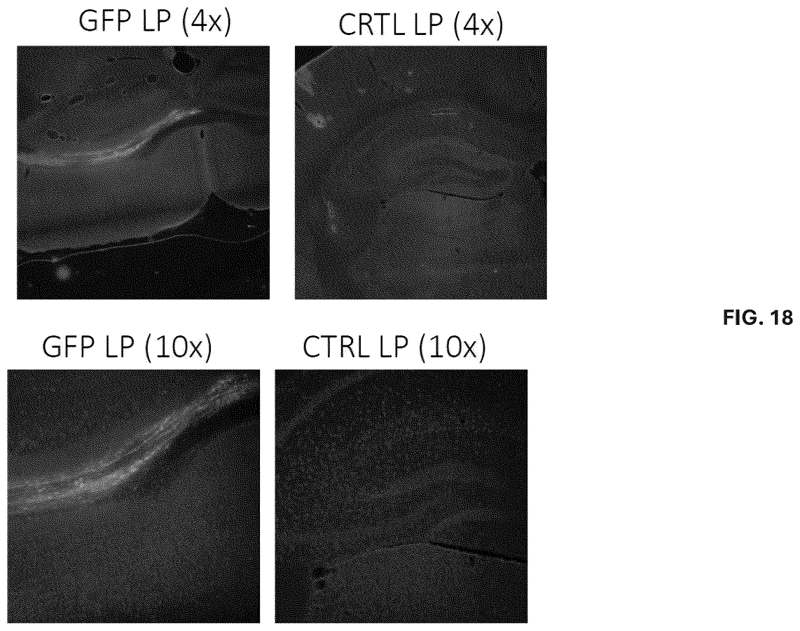

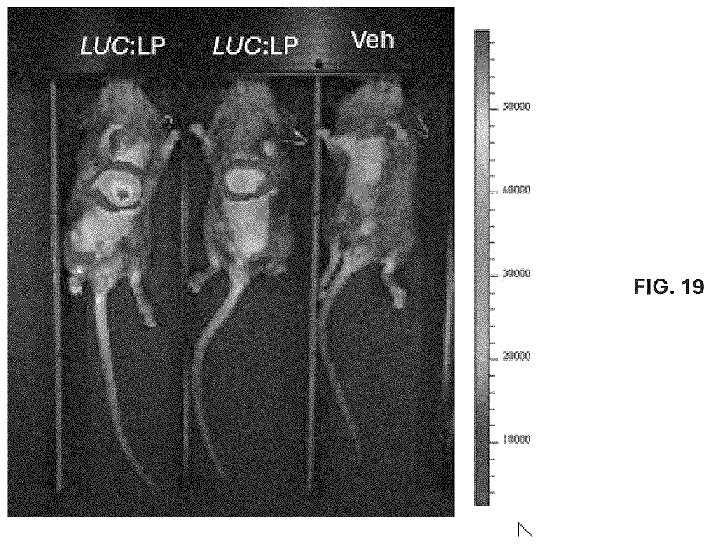

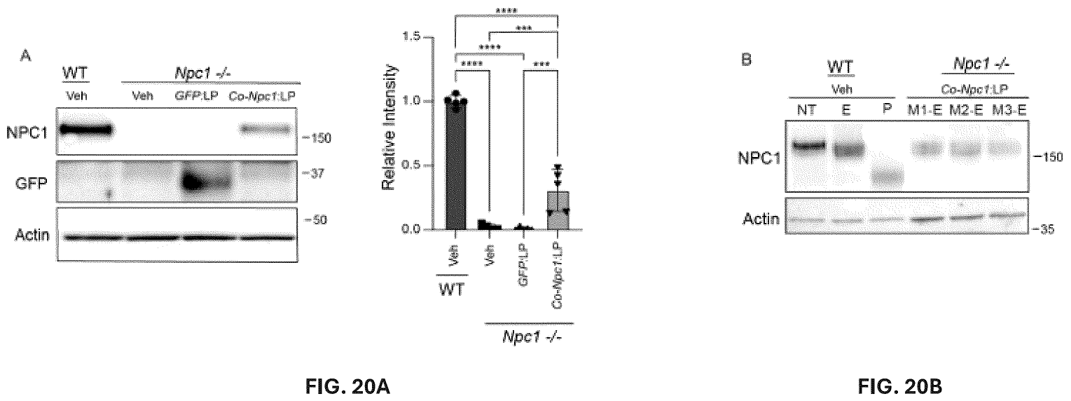

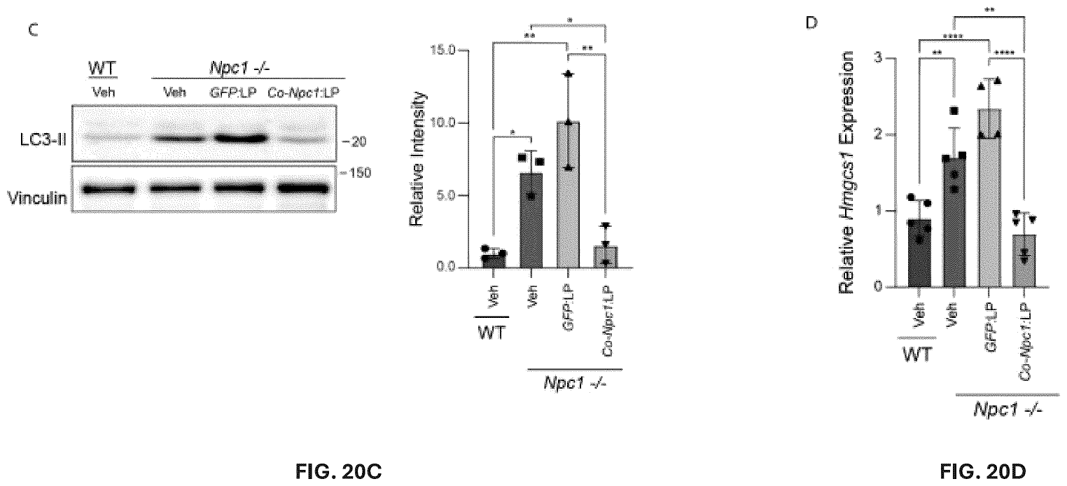

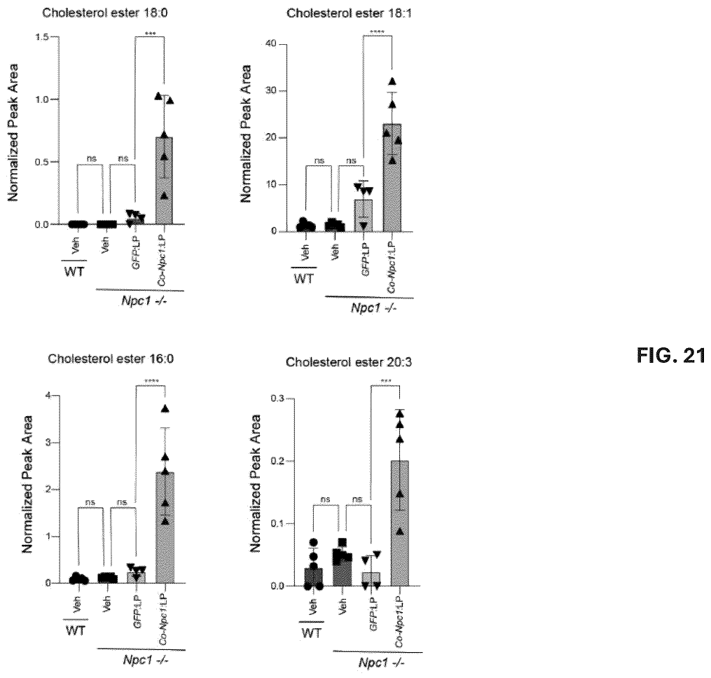

### DESCRIPTION

Niemann-Pick disease type C (NPC) is a rare, progressive genetic disorder caused by mutations in the NPC1 or NPC2 genes, leading to impaired intracellular cholesterol and lipid transport. This dysfunction results in the accumulation of lipids within late endosomes and lysosomes, causing widespread cellular damage and manifesting as severe neurological and systemic symptoms. Current therapeutic approaches for NPC are limited, with no widely available curative treatments. Gene therapy has been explored as a potential solution, but it presents significant challenges, including insertional mutagenesis, immune responses to viral vectors, and limited transduction efficiency. Additionally, the large size of the NPC1 mRNA has historically hindered the development of effective delivery systems, leaving an unmet need for safe and efficient therapeutic options.

The present disclosure addresses these limitations by providing lipid nanoparticles (LNPs) encapsulating mRNA sequences coding for NPC1 and/or NPC2 proteins. These LNPs utilize a lipid formulation, such as one comprising SM-102, DMG-PEG 2000, DSPC, and cholesterol, which has demonstrated safety and efficacy in mRNA-based vaccines. The described composition incorporates codon optimization and optional mRNA modifications, such as N1-methylpseudouridine, 5′ cap, and polyA tail, to enhance mRNA stability and translation efficiency. The LNPs can be prepared using microfluidic mixing, ensuring high encapsulation efficiency, homogeneity, and particle sizes ranging from about 80 to 150 nanometers. This formulation achieves near-complete transfection efficiency across multiple cell types and restores NPC1 protein levels, trafficking, and function in NPC1-deficient cells, as evidenced by correction of cholesterol accumulation and autophagy defects.

By leveraging mRNA-based therapeutics and advanced LNP technology, the described approach provides a safer alternative to gene therapy, with the ability to halt treatment if adverse effects arise. Furthermore, the use of codon-optimized mRNA and the lipid formulation addresses prior challenges associated with the large size of NPC1 mRNA, demonstrating a meaningful advancement in the treatment of Niemann-Pick disease type C. This methodology not only responds to the long-standing need for effective NPC therapies but also offers a scalable, non-immunogenic, and affordable solution for patients suffering from this debilitating condition.

To overcome NPC-loss-of-functions, provided herein are lipid nanoparticles containing mRNA coding for NPC1 and/or NPC2, which upon administration restores the cellular NPC1 and/or NPC2 protein, trafficking, and function providing a treatment for Niemann-Pick C disease.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, several embodiments with regards to methods and materials are described herein. As used herein, each of the following terms has the meaning associated with it in this section.

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

For the purposes of clarity and a concise description, features can be described herein as part of the same or separate embodiments; however, it will be appreciated that the scope of the invention may include embodiments having combinations of all or some of the features described.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

As used herein, the indefinite articles “a,” “an” and “the” should be understood to include plural reference unless the context clearly indicates otherwise.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

As used herein, the term “about” means plus or minus 10% of the indicated value. For example, about 100 means from 90 to 110. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

“Homologous” or “identity” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating,” “treat” and “treatment” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

The term “delivery vehicle” or “carrier” refers to any kind of device or material which can be used to deliver the invention in vivo or can be added to a composition comprising RNA and/or lipids administered to an animal.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

As used herein “injecting, administering or applying” includes administration of the invention by any number of routes and means including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, or rectal means.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

As used herein, the term “pharmaceutically acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application. As used herein, “pharmaceutical compositions” include formulations for human and veterinary use.

The terms “patient,” “individual,” or “subject” are used interchangeably herein, and refer to a mammal, particularly, a human. As used herein, a “subject in need thereof” is a patient, animal, mammal, or human, who will benefit from the method of this invention. The patient may have mild, intermediate or severe disease, or may only experience transient, or fluctuating symptoms. The patient may be an individual, at risk of developing a disease, in need of treatment or in need of diagnosis based on particular symptoms or family history. In some cases, the terms may refer to treatment of animals, in such as veterinary applications (e.g., companion animals (e.g., cats or dogs) or livestock (e.g., pigs, cows, horses)).

A disease, condition, or disorder is “alleviated” if the severity of a symptom of the disease or disorder, the frequency with which such a symptom is experienced by a subject, or both, are reduced. As used herein, the terms “indicates” or “correlates” (or “indicating” or “correlating,” or “indication” or “correlation,” depending on the context) in reference to a parameter, e.g., a modulated proportion, level, or cellular localization in a sample from a patient, may mean that the patient has or at risk of having Niemann Pick Type C Disease.

Various methodologies of the instant invention include a step that involves comparing a value, level, feature, characteristic, property, etc. to a “suitable control,” referred to interchangeably herein as an “appropriate control” or a “control sample.” A “suitable control,” “appropriate control” or a “control sample” is any control or standard familiar to one of ordinary skill in the art useful for comparison purposes. In one embodiment, a “suitable control” or “appropriate control” is a value, level, feature, characteristic, property, etc., determined in a cell, organ, or patient, e.g., a control or normal cell, organ, or patient, exhibiting, for example, normal traits. For example, the biomarkers of the present invention may be assayed in a sample from an unaffected individual (UI) or a normal control individual (NC) (both terms are used interchangeably herein) or other affected individual. In another embodiment, a “suitable control” or “appropriate control” is a value, level, feature, characteristic, property, etc. determined prior to performing a therapy on a patient. In yet another embodiment, a transcription rate, mRNA level, translation rate, protein level, biological activity, cellular characteristic or property, genotype, phenotype, etc. can be determined prior to, during, or after administering a therapy into a cell, organ, or patient. In a further embodiment, a “suitable control” or “appropriate control” is a predefined value, level, feature, characteristic, property, etc.

The terms “sample,” “patient sample,” “biological sample,” and the like, encompass a variety of sample types obtained from a patient, individual, or subject and can be used in a diagnostic or monitoring assay. The patient sample may be obtained from a healthy subject, a diseased patient or a patient having associated symptoms. Moreover, a sample obtained from a patient can be divided and only a portion may be used for diagnosis. Further, the sample, or a portion thereof, can be stored under conditions to maintain sample for later analysis.

The term “otherwise identical sample,” as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

The terms “measuring” and “determining” are used interchangeably throughout and refer to methods which include obtaining a patient sample and/or detecting the level of a protein, nucleic acid or cellular component or biomarker(s) in a sample. For example, in one embodiment, the terms refer to obtaining a patient sample and detecting the level of one or more biomarkers in the sample. In another embodiment, the terms “measuring” and “determining” mean detecting level of one or more biomarkers in a patient sample. Measuring can be accomplished by methods known in the art and those further described herein including. The term “measuring” is also used interchangeably throughout with the term “detecting.”

As used herein, an “instructional material” includes a publication, a recording, a diagram, or any other medium of expression which can be used to communicate the usefulness of the of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified invention or be shipped together with a container. Alternatively, the instructional material may be shipped separately from the container with the intention that the instructional material and the invention be used cooperatively by the recipient.

As used herein, the terms “including”, “includes”, “having”, “has”, “with”, or variants thereof, are intended to be inclusive similar to the term “comprising.”

The terms “comprises,” “comprising,” and the like can have the meaning ascribed to them in U.S. Patent Law and can mean “includes,” “including” and the like. As used herein, “including” or “includes” or the like means including, without limitation.

Methods involving conventional molecular biology techniques are described herein. Such techniques are generally known in the art and are described in detail in methodology treatises, such as Molecular Cloning: A Laboratory Manual, Sambrook et al., Cold Spring Harbor Laboratory Press, Cold Spring Harbor, N.Y.

Without further elaboration, it is believed that one skilled in the art, using the preceding description, can utilize the present invention to the fullest extent. The following examples are illustrative only, and not limiting of the remainder of the disclosure in any way whatsoever.

### Neiman Pick Type C Disease

Niemann-Pick disease, type C1 (NPC1) is a membrane protein that mediates intracellular cholesterol trafficking in mammals. In humans the protein is encoded by the NPC1 gene (chromosome location 18q11).

NPC1 was identified as the gene that when mutated, results in Niemann-Pick disease, type C. Niemann-Pick disease, type C is a rare neurovisceral lipid storage disorder resulting from autosomal recessively inherited loss-of-function mutations in either NPC1 or NPC2. This disrupts intracellular lipid transport, leading to the accumulation of lipid products in the late endosomes and lysosomes. Approximately 95% of NPC patients are found to have mutations in the NPC1 gene.

Niemann-Pick type C has a wide clinical spectrum. Affected individuals may have enlargement of the spleen (splenomegaly) and liver (hepatomegaly), or enlarged spleen or liver combined (hepatosplenomegaly), but this finding may be absent in later onset cases. Prolonged jaundice or elevated bilirubin can present at birth. In some cases, however, enlargement of the spleen or liver does not occur for months or years—or not at all. Enlargement of the spleen or liver frequently becomes less apparent with time, in contrast to the progression of other lysosomal storage diseases such as Niemann-Pick disease, Types A and B or Gaucher disease.

Progressive neurological disease is the hallmark of Niemann-Pick type C disease and is responsible for disability and premature death in all cases beyond early childhood. Classically, children with NPC may initially present with delays in reaching normal developmental milestones skills before manifesting cognitive decline (dementia).

Neurological signs and symptoms include cerebellar ataxia (unsteady walking with uncoordinated limb movements), dysarthria (slurred speech), dysphagia (difficulty in swallowing), tremor, epilepsy (both partial and generalized), vertical supranuclear palsy (upgaze palsy, downgaze palsy, saccadic palsy or paralysis), sleep inversion, gelastic cataplexy (sudden loss of muscle tone or drop attacks), dystonia (abnormal movements or postures caused by contraction of agonist and antagonist muscles across joints), most commonly begins with inturning of one foot when walking (action dystonia) and may spread to become generalized, spasticity (velocity dependent increase in muscle tone), hypotonia, ptosis (drooping of the upper eyelid), microcephaly (abnormally small head), psychosis, progressive dementia, progressive hearing loss, bipolar disorder, major and psychotic depression that can include hallucinations, delusions, mutism, or stupor.

In the terminal stages of Niemann-Pick type C disease, the patient is bedridden, with complete ophthalmoplegia, loss of volitional movement and severe dementia.

### NPC1/NPC2

Approximately 95% of Niemann-Pick type C cases are caused by genetic mutations in the NPC1 gene, referred to as type C1; 5% are caused by mutations in the NPC2 gene, referred to as type C2. The clinical manifestations of types Niemann-Pick types C1 and C2 are similar because the respective genes are both involved in egress of lipids, particularly cholesterol, from late endosomes or lysosomes. The NPC1 gene is located on chromosome 18 (18q11-q12).

Human NPC1 mRNA and Amino Acid Sequence (NCBI Reference Sequence: NM_000271.5 and NCBI Reference Sequence: NP_000262.2)

MTARGLALGLLLLLLCPAQVFSQSCVWYGECGIAYGDKRYNCEYSG
PPKPLPKDGYDLVQELCPGFFFGNVSLCCDVRQLQTLKDNLQLPL
QFLSRCPSCFYNLLNLFCELTCSPRQSQFLNVTATEDYVDPVTNQ
TKTNVKELQYYVGQSFANAMYNACRDVEAPSSNDKALGLLCGKDA
DACNATNWIEYMFNKDNGQAPFTITPVFSDFPVHGMEPMNNATKG
CDESVDEVTAPCSCQDCSIVCGPKPQPPPPPAPWTILGLDAMYVI
MWITYMAFLLVFFGAFFAVWCYRKRYFVSEYTPIDSNIAFSVNAS
DKGEASCCDPVSAAFEGCLRRLFTRWGSFCVRNPGCVIFFSLVFI
TACSSGLVFVRVTTNPVDLWSAPSSQARLEKEYFDQHFGPFFRTE
QLIIRAPLTDKHIYQPYPSGADVPFGPPLDIQILHQVLDLQIAIE
NITASYDNETVTLQDICLAPLSPYNTNCTILSVLNYFQNSHSVLD
HKKGDDFFVYADYHTHFLYCVRAPASLNDTSLLHDPCLGTFGGPV
FPWLVLGGYDDQNYNNATALVITFPVNNYYNDTEKLQRAQAWEKE
FINFVKNYKNPNLTISFTAERSIEDELNRESDSDVFTVVISYAIM
FLYISLALGHMKSCRRLLVDSKVSLGIAGILIVLSSVACSLGVFS
YIGLPLTLIVIEVIPFLVLAVGVDNIFILVQAYQRDERLQGETLD
QQLGRVLGEVAPSMFLSSFSETVAFFLGALSVMPAVHTFSLFAGL
AVFIDELLQITCFVSLLGLDIKRQEKNRLDIFCCVRGAEDGTSVQ
ASESCLFRFFKNSYSPLLLKDWMRPIVIAIFVGVLSFSIAVLNKV
DIGLDQSLSMPDDSYMVDYFKSISQYLHAGPPVYFVLEEGHDYTS
SKGQNMVCGGMGCNNDSLVQQIFNAAQLDNYTRIGFAPSSWIDDY
FDWVKPQSSCCRVDNITDQFCNASVVDPACVRCRPLTPEGKQRPQ
GGDFMRFLPMFLSDNPNPKCGKGGHAAYSSAVNILLGHGTRVGAT
YFMTYHTVLQTSADFIDALKKARLIASNVTETMGINGSAYRVFPY
SVFYVFYEQYLTIIDDTIFNLGVSLGAIFLVTMVLLGCELWSAVI
MCATIAMVLVNMFGVMWLWGISLNAVSLVNLVMSCGISVEFCSHI
TRAFTVSMKGSRVERAEEALAHMGSSVFSGITLTKFGGIVVLAFA
KSQIFQIFYFRMYLAMVLLGATHGLIFLPVLLSYIGPSVNKAKSC
ATEERYKGTERERLLNF

(SEQ ID NO: 1), a different isoform of the protein, or a polypeptide having at least 80%, 82%, 85%, 86%, 88%, 90%, 92%, 94%, 95%, 97%, 98% or 99% amino acid sequence identity thereto, or a gene comprising or RNA corresponding to (accession number NM_000271):

| | |
|---|---|
| 1 | cttcctgacc ggcgcgcgca gcctgctgcc gcggtcagcg cctgctcctg ctcctccgct |
| 61 | cctcctgcgc ggggtgctga aacagcccgg ggaagtagag ccgcctccgg ggagcccaac |
| 121 | cagccgaacg ccgccggcgt cagcagcctt gegcggccac agcatgaccg ctcgcggcct |
| 181 | ggcccttggc ctcctcctgc tgctactgtg tccagcgcag gtgttttcac agtcctgtgt |
| 241 | ttggtatgga gagtgtggaa ttgcatatgg ggacaagagg tacaattgcg aatattctgg |
| 301 | cccaccaaaa ccattgccaa aggatggata tgacttagtg caggaactct gtccaggatt |
| 361 | cttctttggc aatgtcagtc tctgttgtga tgttcggcag cttcagacac taaaagacaa |
| 421 | cctgcagctg cctctacagt ttctgtccag atgtccatcc tgtttttata acctactgaa |
| 481 | cctgttttgt gagctgacat gtagccctcg acagagtcag tttttgaatg ttacagctac |
| 541 | tgaagattat gttgatcctg ttacaaacca gacgaaaaca aatgtgaaag agttacaata |
| 601 | ctacgtcgga cagagttttg ccaatgcaat gtacaatgcc tgccgggatg tggaggcccc |
| 661 | ctcaagtaat gacaaggccc tgggactcct gtgtgggaag gacgctgacg cctgtaatgc |
| 721 | caccaactgg attgaataca tgttcaataa ggacaatgga caggcacctt ttaccatcac |
| 781 | tcctgtgttt tcagattttc cagtccatgg gatggagccc atgaacaatg ccaccaaagg |
| 841 | ctgtgacgag tctgtggatg aggtcacagc accatgtagc tgccaagact gctctattgt |
| 901 | ctgtggcccc aagccccagc ccccacctcc tcctgctccc tggacgatcc ttggcttgga |
| 961 | cgccatgtat gtcatcatgt ggatcaccta catggcgttt ttgcttgtgt tttttggagc |
| 1021 | attttttgca gtgtggtgct acagaaaacg gtattttgtc tccgagtaca ctcccatoga |
| 1081 | tagcaatata gctttttctg ttaatgcaag tgacaaagga gaggcgtcct gctgtgaccc |
| 1141 | tgtcagcgca gcatttgagg gctgcttgag gcggctgttc acacgctggg ggtctttctg |
| 1201 | cgtccgaaac cctggctgtg tcattttctt ctcgctggtc ttcattactg cgtgttcgtc |
| 1261 | aggcctggtg tttgtccggg tcacaaccaa tccagttgac ctctggtcag cccccagcag |
| 1321 | ccaggctcgc ctggaaaaag agtactttga ccagcacttt gggcctttct tccggacgga |
| 1381 | gcagctcatc atccgggccc ctctcactga caaacacatt taccagccat acccttcggg |
| 1441 | agctgatgta ccctttggac ctccgcttga catacagata ctgcaccagg ttcttgactt |
| 1501 | acaaatagcc atcgaaaaca ttactgcctc ttatgacaat gagactgtga cacttcaaga |
| 1561 | catctgcttg gcccctcttt caccgtataa cacgaactgc accattttga gtgtgttaaa |
| 1621 | ttacttccag aacagccatt ccgtgctgga ccacaagaaa ggggacgact tctttgtgta |
| 1681 | tgccgattac cacacgcact ttctgtactg cgtacgggct cctgcctctc tgaatgatac |
| 1741 | aagtttgctc catgaccctt gtctgggtac gtttggtgga ccagtgttcc cgtggcttgt |
| 1801 | gttgggaggc tatgatgatc aaaactacaa taacgccact gcccttgtga ttaccttccc |
| 1861 | tgtcaataat tactataatg atacagagaa gctccagagg gcccaggcct gggaaaaaga |
| 1921 | gtttattaat tttgtgaaaa actacaagaa toccaatctg accatttcct tcactgctga |
| 1981 | acgaagtatt gaagatgaac taaatcgtga aagtgacagt gatgtcttca ccgttgtaat |
| 2041 | tagctatgcc atcatgtttc tatatatttc cctagccttg gggcacatga aaagctgtcg |
| 2101 | caggcttctg gtggattcga aggtctcact aggcatcgcg ggcatcttga tcgtgctgag |
| 2161 | ctcggtggct tgctccttgg gtgtcttcag ctacattggg ttgcccttga ccctcattgt |
| 2221 | gattgaagtc atcccgttcc tggtgctggc tgttggagtg gacaacatct tcattctggt |
| 2281 | gcaggcctac cagagagatg aacgtcttca aggggaaacc ctggatcagc agctgggcag |
| 2341 | ggtcctagga gaagtggctc ccagtatgtt cctgtcatcc ttttctgaga ctgtagcatt |
| 2401 | tttcttagga gcattgtccg tgatgccagc cgtgcacacc ttctctctct ttgcgggatt |
| 2461 | ggcagtcttc attgactttc ttctgcagat tacctgtttc gtgagtctct tggggttaga |
| 2521 | cattaaacgt caagagaaaa atcggctaga catcttttgc tgtgtcagag gtgctgaaga |
| 2581 | tggaacaagc gtccaggcct cagagagctg tttgtttcgc ttcttcaaaa actcctattc |
| 2641 | tccacttctg ctaaaggact ggatgagacc aattgtgata gcaatatttg tgggtgttct |
| 2701 | gtcattcagc atcgcagtcc tgaacaaagt agatattgga ttggatcagt ctctttcgat |
| 2761 | gccagatgac tcctacatgg tggattattt caaatccatc agtcagtacc tgcatgcggg |
| 2821 | tccgcctgtg tactttgtcc tggaggaagg gcacgactac acttcttcca aggggcagaa |
| 2881 | catggtgtgc ggcggcatgg gctgcaacaa tgattccctg gtgcagcaga tatttaacgc |
| 2941 | ggcgcagctg gacaactata cccgaatagg cttcgccccc tcgtcctgga tcgacgatta |
| 3001 | tttcgactgg gtgaagccac agtcgtcttg ctgtcgagtg gacaatatca ctgaccagtt |
| 3061 | ctgcaatgct tcagtggttg accctgcctg cgttcgctgc aggcctctga ctccggaagg |
| 3121 | caaacagagg cctcaggggg gagacttcat gagattcctg cccatgttcc tttcggataa |
| 3181 | ccctaacccc aagtgtggca aagggggaca tgctgcctat agttctgcag ttaacatcct |
| 3241 | ccttggccat ggcaccaggg tcggagccac gtacttcatg acctaccaca ccgtgctgca |
| 3301 | gacctctgct gactttattg acgctctgaa gaaagcccga cttatagcca gtaatgtcac |
| 3361 | cgaaaccatg ggcattaacg gcagtgccta ccgagtattt ccttacagtg tgttttatgt |
| 3421 | cttctacgaa cagtacctga ccatcattga cgacactatc ttcaacctcg gtgtgtccct |
| 3481 | gggcgcgata tttctggtga ccatggtcct cctgggctgt gagctctggt ctgcagtcat |
| 3541 | catgtgtgcc accatcgcca tggtcttggt caacatgttt ggagttatgt ggctctgggg |
| 3601 | catcagtctg aacgctgtat ccttggtcaa cctggtgatg agctgtggca tctccgtgga |
| 3661 | gttctgcagc cacataacca gagcgttcac ggtgagcatg aaaggcagcc gcgtggagcg |
| 3721 | cgcggaagag gcacttgccc acatgggcag ctccgtgttc agtggaatca cacttacaaa |
| 3781 | atttggaggg attgtggtgt tggcttttgc caaatctcaa attttccaga tattctactt |
| 3841 | caggatgtat ttggccatgg tcttactggg agccactcac ggattaatat ttctccctgt |
| 3901 | cttactcagt tacatagggc catcagtaaa taaagccaaa agttgtgcca ctgaagagcg |
| 3961 | atacaaagga acagagcgcg aacggcttct aaatttctag ccctctcgca gggcatcctg |
| 4021 | actgaactgt gtctaagggt cggtcggttt accactggac gggtgctgca tcggcaaggc |
| 4081 | caagttgaac accggatggt gccaaccatc ggttgtttgg cagcagcttt gaacgtagcg |
| 4141 | cctgtgaact caggaatgca cagttgactt gggaagcagt attactagat ctggaggcaa |
| 4201 | ccacaggaca ctaaacttct cccagcctct tcaggaaaga aacctcattc tttggcaagc |
| 4261 | aggaggtgac actagatggc tgtgaatgtg atccgctcac tgacactctg taaaggccaa |
| 4321 | tcaatgcact gtctgtctct ccttttagga gtaagccatc ccacaagttc tataccatat |
| 4381 | ttttagtgac agttgaggtt gtagatacac tttataacat tttatagttt aaagagcttt |
| 4441 | attaatgcaa taaattaact ttgtacacat ttttatataa aaaaacagca agtgatttca |
| 4501 | gaatgttgta ggcctcatta gagcttggtc tccaaaaatc tgtttgaaaa aagcaacatg |
| 4561 | ttcttcacag tgttccccta gaaaggaaga gatttaattg ccagttagat gtggcatgaa |
| 4621 | atgagggaca aagaaagcat ctcgtaggtg tgtctactgg gttttaactt atttttcttt |
| 4681 | aataaaatac attgttttcc taagttttgg ggttacccta tctgctttga gagacaaata |
| 4741 | caaaagctaa atggaagaga |

(SEQ ID NO: 2), a different isoform of the RNA, or a nucleic acid having at least 80%, 82%, 85%, 86%, 88%, 90%, 92%, 94%, 95%, 97%, 98% or 99% nucleic acid sequence identity thereto.
   Human NPC2 mRNA and Amino Acid Sequence (NCBI Reference Sequence: NM_006432.5)

MRFLAATFLLLALSTAAQAEPVQFKDCGSVDGVIKEVNVSPCPT
QPCQLSKGQSYSVNVTFTSNIQSKSSKAVVHGILMGVPVPFPIP
EPDGCKSGINCPIQKDKTYSYLNKLPVKSEYPSIKLVVEWQLQD
DKNOSLFCWEIPVQIVSHL

(SEQ ID NO: 3), a different isoform of the protein, or a polypeptide having at least 80%, 82%, 85%, 86%, 88%, 90%, 92%, 94%, 95%, 97%, 98% or 99% amino acid sequence identity thereto, or a gene comprising or RNA corresponding to (NCBI Reference Sequence: NM_006432.5)

| | |
|---|---|
| 1 | ctttcccgag cttggaactt cgttatccgc |
| | gatgcgtttc ctggcagcta cattcctgct |
| 61 | cctggcgctc agcaccgctg cccaggccga |
| | accggtgcag ttcaaggact gcggttctgt |
| 121 | ggatggagtt ataaaggaag tgaatgtgag |
| | cccatgcccc acccaaccct gccagctgag |
| 181 | caaaggacag tcttacagcg tcaatgtcac |
| | cttcaccagc aatattcagt ctaaaagcag |
| 241 | caaggccgtg gtgcatggca tcctgatggg |
| | cgtcccagtt ccctttccca ttcctgagcc |
| 301 | tgatggttgt aagagtggaa ttaactgccc |
| | tatccaaaaa gacaagacct atagctacct |
| 361 | gaataaacta ccagtgaaaa gcgaatatcc |
| | ctctataaaa ctggtggtgg agtggcaact |
| 421 | tcaggatgac aaaaaccaaa gtctcttctg |
| | ctgggaaatc ccagtacaga tcgtttctca |
| 481 | tctctaagtg cctcattgag ttcggtgcat |
| | ctggccaatg agtctgctga gactcttgac |
| 541 | agcacctcca gctctgctgc ttcaacaaca |
| | gtgacttgct ctccaatggt atccagtgat |
| 601 | tcgttgaaga ggaggtgctc tgtagcagaa |
| | actgagctcc gggtggctgg ttctcagtgg |
| 661 | ttgtctcatg tctctttttc tgtcttaggt |
| | ggtttcatta aatgcagcac ttggttagca |
| 721 | gatgtttaat ttttttttta acaacattaa |
| | cttgtggcct ctttctacac ctggaaattt |
| 781 | actcttgaat aaataaaaac tcgtttgtct |
| | tgtcttctgc a |

(SEQ ID NO: 4), a different isoform of the RNA, or a nucleic acid having at least 80%, 82%, 85%, 86%, 88%, 90%, 92%, 94%, 95%, 97%, 98% or 99% nucleic acid sequence identity thereto.

### Lipid Nanoparticles

Provided herein are lipid nanoparticles (LPNs) containing NPC1 and/or NPC2 mRNA. The mRNA can be modified. Such modifications include codon optimization (such as corresponding to tRNA frequency of the species being treated, e.g., human) and other modifications available to an art worker to make the RNA more stable or increase translation. The particles are generated by microfluidic mixing and are composed of the Moderna COVID vaccine lipid formulation (SM-102, DMG-PEG 2000, DSPC and cholesterol).

SM-102 (9-Heptadecanyl 8-{(2-hydroxyethyl)[6-oxo-6-(undecyloxy)hexyl]amino}octanoate; CAS No. 2089251-47-6) is a synthetic amino lipid which is used in combination with other lipids to form lipid nanoparticles. These can be used for the delivery of mRNA, including mRNA-based vaccines, and in particular SM-102 forms part of the drug delivery system for the Moderna COVID-19 vaccine.

DMP-PEG 2000 (1,2-Dimyristoyl-sn-glycero-3-methoxypolyethylene glycol; CAS No. 1397695-86-1) is a synthetic lipid formed by the PEGylation of myristoyl diglyceride. It is used to manufacture lipid nanoparticles that are used in, for example, mRNA vaccines, and in particular forms part of the drug delivery system for the Moderna COVID-19 vaccine.

DSPC-Distearoylphosphatidylcholine ((2R)-2,3-Bis(octadecanoyloxy) propyl 2-(trimethylazaniumyl)ethyl phosphate; CAS No. 4539-70-2 (racemic); 816-94-4 (R enantiomer)) is a phosphatidylcholine, a phospholipid. It is a natural constituent of cell membranes. It can be used to prepare lipid nanoparticles which are used in, for example, mRNA vaccines. In particular, it forms part of the drug delivery system for the Moderna and Pfizer COVID-19 vaccines.

The LPNs provided herein are non-immunogenic, affordable to generate, and readily transfect multiple cell types.

To stabilize the NPC1 or NPC2 mRNA and increase protein production the RNAs can be codon optimized optionally include one or more of N1-methylpseudouridine, 5′ cap and a polyA tail, each of which can increase RNA half-life.

These particles demonstrated 100% transfection efficiency and showed complete restoration of NPC1 protein levels in NPC1−/− deficient cells, as well as restoration of processing and trafficking as shown via endoglycosidase H assay and function as assessed by correction of cholesterol.

Although there has been some work on the development of gene therapies for loss-of-function diseases, there remains significant hurdles, including the introduction of genetic mutations, transduction capacity and viral based immune responses. By relying on mRNA LNPs this invention is significantly safer than gene therapy and treatment can stop if adverse effects are discovered in a patient. The Moderna particles have been well tolerated in the over ˜230 million doses which make this formulation particularly safe. Although mRNA has been proposed as a therapeutic for NPC1, the large size of the mRNA has prevented other groups from successfully developing a particle which corrects Niemann-Pick C cellular phenotypes. Thus, demonstrating a long felt need in the treatment of Niemann-Pick type C. Surprisingly, it was found that the Moderna formulation readily encapsulates the NPC1 mRNA and does not significantly alter particle size when compared to a GFP mRNA report particle.

For the data provided herein, CleanCap® EGFP mRNA (L-7601), NPC1 mRNA, and codon optimized NPC1 mRNA were obtained from TriLink Biotechnologies. SM-102 (MW 710.2) (33474) and Cholesterol (MW 386.65) (C8667) were purchased from Cayman Chemical and Sigma Aldrich, respectively. 1,2-distearoyl-sn-glycero-3-phosphocholine (DSPC, 18:0 PC; MW 790.2) (850365P) and 1,2-dimyristoyl-rac-glycero-3-methoxypolyethylene glycol-2000 (DMG-PEG2000; MW 2526) (880151P) were purchased from Avanti Polar Lipids. Quant-iT™ RiboGreen™ RNA Assay Kit (R11490) was sourced from ThermoFisher Scientific.

In one embodiment, SM-102, Cholesterol, DSPC and DMG-PEG2000 were dissolved in ethanol and mixed at the molar ratio of 50:38.5:10:1.5 (lipidoids:cholesterol:helper lipids:peg lipids in a particle). EGFP mRNA, NPC1 mRNA, or codon optimized NPC1 mRNA was dissolved in a 25 mM sodium acetate buffer, pH 4.0 to achieve a concentration of 140 ng/μL. The mRNA solution and the lipid solution were mixed at a 3:1 volume ratio with a total flow rate of 3 mL/min to achieve N:P ratio (molar ratio of amine groups on SM-102:phosphates on the mRNA) of 6:1 using a microfluidic mixing device from Precigenome. The resultant LNPs was buffer exchanged into pH7.4 1×PBS using 10 kDa molecular weight cut off centrifugal tubes. The size and polydispersity index (PdI) of the particles was measured using dynamic light scattering (DLS). The encapsulation efficiency of the mRNA was calculated using a modified Quanti-Ti Ribogreen RNA assay.

In one embodiment, The N:P ratio (molar ratio of amine groups on SM-102:phosphates on the mRNA) was set to be at 6:1. Given that SM-102 lipid contains one tertiary amine and the oligonucleotide contains one phosphate, the desired N:P ratio can be attained.

Using a modified Quanti-Ti Ribogreen RNA assay, the empirically determined concentration was 80-90 ng/μL. (The theoretical encapsulated mRNA concentration in the LNP is 105 ng/μL.) LNPs can be concentrated to achieve an mRNA concentration of, for example, 100 ng/μL.

### Treatment/Administration

Niemann-Pick type C is diagnosed by assaying cultured fibroblasts for cholesterol esterification and staining for unesterified cholesterol with filipin. The fibroblasts are grown from a small skin biopsy taken from a patient with suspected NPC. The diagnosis can be confirmed by identifying mutations in the NPC1 or NPC2 genes in 80-90% of cases.

In some embodiments, RNA coding for NPC1 and/or NPC2 are administered to a subject.

The administration can be local by, for example, injection or with the use of a catheter or during surgery. The administration can also be oral, intravenous or subcutaneous.

Pharmaceutically acceptable carriers include physiologically tolerable or acceptable diluents, excipients, solvents or adjuvants. The compositions are preferably sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, lipids/liposomes, lipid nanoparticles or mixtures of these substances, and the like.

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g. 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Pharmaceutical compositions according to the present invention can be prepared in a manner fully within the skill of the art.

All publications, patents, and patent applications mentioned in this specification are herein incorporated by reference to the same extent as if each individual publication, patent, or patent application was specifically and individually indicated to be incorporated by reference. In the event that the definition of a term incorporated by reference conflicts with a term defined herein, this specification shall control.

What is claimed is:

1. A composition comprising lipid nanoparticles (LNPs) encapsulating a mRNA sequence coding for NPC1 protein and/or NPC2 protein, wherein the LPNs comprise a combination of SM-102, DMG-PEG 2000, DSPC and cholesterol.

2. The composition of claim 1, wherein the mRNA is codon optimized.

3. The composition of claim 1, wherein the mRNA comprises one or more of N1-methylpseudouridine, 5′ cap or a polyA tail.

4. The composition of claim 1, wherein in the NPC1 is coded for by SEQ ID NO: 2 or a nucleic acid having at least 80% identity thereto.

5. The composition of claim 1, wherein the NPC2 is coded for by SEQ ID NO: 4 or a nucleic acid having at least 80% identity thereto.

6. The composition of claim 1, wherein the lipids are present in the ratio of 50:38.5:10:1.5 (lipidoids:cholesterol:helper lipids:peg lipids) in a LNP.

7. The composition of claim 1, further comprising a carrier.

8. The composition of claim 7, wherein the carrier is water, saline, and phosphate buffered or saline.

9. The composition of claim 1, wherein the lipid nanoparticles are prepared:
by microfluidic mixing.

10. The composition according to claim 1, wherein the lipid nanoparticles have an average particle diameter ranging from about 80 to 150 nanometers.

11. A method to treat Niemann-Pick disease, type C1 comprising administering the composition of claim 1 to a subject in need thereof.

12. The method of claim 11, wherein the mRNA is codon optimized.

13. The method of claim 11, wherein the mRNA comprises one or more of N1-methylpseudouridine, 5′ cap or a polyA tail.

14. The method of claim 11, wherein in the NPC1 is coded for by SEQ ID NO: 2 or a nucleic acid having at least 80% identity thereto.

15. The method of claim 11, wherein the NPC2 is coded for by SEQ ID NO: 4 or a nucleic acid having at least 80% identity thereto.

16. The method of claim 11, wherein the lipids are present in the ratio of 50:38.5:10:1.5 (lipidoids:cholesterol:helper lipids:peg lipids) in a LNP.

17. The method of claim 1, further comprising a carrier.

18. The method of claim 11, wherein the treatment results in the decrease of one or more symptoms of Niemann-Pick disease type C1.

19. The method of claim 18, wherein the one or more symptoms include cerebellar ataxia (unsteady walking with uncoordinated limb movements), dysarthria (slurred speech), dysphagia (difficulty in swallowing), tremor, epilepsy (both partial and generalized), vertical supranuclear palsy (upgaze palsy, downgaze palsy, saccadic palsy or paralysis), sleep inversion, gelastic cataplexy (sudden loss of muscle tone or drop attacks), dystonia (abnormal movements or postures caused by contraction of agonist and antagonist muscles across joints), inturning of one foot when walking (action dystonia), spasticity (velocity dependent increase in muscle tone), hypotonia, ptosis (drooping of the upper eyelid), microcephaly (abnormally small head), psychosis, dementia, hearing loss, bipolar disorder, major and psychotic depression that can include hallucinations, delusions, mutism, stupor, or altered liver function.

20. The method of claim 11, wherein the treatment results in an increased level of NPC1 protein.


SEQUENCE LISTING (4 sequences)

SEQ ID NO: 1 - protein, 1278 aa, Homo sapiens
MTARGLALGLLLLLLCPAQVFSQSCVWYGECGIAYGDKRYNCEYSGPPKPLPKDGYDLVQELCPGFFFGNVSLCCDVRQLQTLKDNLQLPLQFLSRCPSCFYNLLNLFCELTCSPRQSQFLNVTATEDYVDPVTNQTKTNVKELQYYVGQSFANAMYNACRDVEAPSSNDKALGLLCGKDADACNATNWIEYMFNKDNGQAPFTITPVFSDFPVHGMEPMNNATKGCDESVDEVTAPCSCQDCSIVCGPKPQPPPPPAPWTILGLDAMYVIMWITYMAFLLVFFGAFFAVWCYRKRYFVSEYTPIDSNIAFSVNASDKGEASCCDPVSAAFEGCLRRLFTRWGSFCVRNPGCVIFFSLVFITACSSGLVFVRVTTNPVDLWSAPSSQARLEKEYFDQHFGPFFRTEQLIIRAPLTDKHIYQPYPSGADVPFGPPLDIQILHQVLDLQIAIENITASYDNETVTLQDICLAPLSPYNTNCTILSVLNYFQNSHSVLDHKKGDDFFVYADYHTHFLYCVRAPASLNDTSLLHDPCLGTFGGPVFPWLVLGGYDDQNYNNATALVITFPVNNYYNDTEKLQRAQAWEKEFINFVKNYKNPNLTISFTAERSIEDELNRESDSDVFTVVISYAIMFLYISLALGHMKSCRRLLVDSKVSLGIAGILIVLSSVACSLGVFSYIGLPLTLIVIEVIPFLVLAVGVDNIFILVQAYQRDERLQGETLDQQLGRVLGEVAPSMFLSSFSETVAFFLGALSVMPAVHTFSLFAGLAVFIDFLLQITCFVSLLGLDIKRQEKNRLDIFCCVRGAEDGTSVQASESCLFRFFKNSYSPLLLKDWMRPIVIAIFVGVLSFSIAVLNKVDIGLDQSLSMPDDSYMVDYFKSISQYLHAGPPVYFVLEEGHDYTSSKGQNMVCGGMGCNNDSLVQQIFNAAQLDNYTRIGFAPSSWIDDYFDWVKPQSSCCRVDNITDQFCNASVVDPACVRCRPLTPEGKQRPQGGDFMRFLPMFLSDNPNPKCGKGGHAAYSSAVNILLGHGTRVGATYFMTYHTVLQTSADFIDALKKARLIASNVTETMGINGSAYRVFPYSVFYVFYEQYLTIIDDTIFNLGVSLGAIFLVTMVLLGCELWSAVIMCATIAMVLVNMFGVMWLWGISLNAVSLVNLVMSCGISVEFCSHITRAFTVSMKGSRVERAEEALAHMGSSVFSGITLTKFGGIVVLAFAKSQIFQIFYFRMYLAMVLLGATHGLIFLPVLLSYIGPSVNKAKSCATEERYKGTERERLLNF

SEQ ID NO: 2 - RNA, 4760 nt, Homo sapiens
  source 1..4760: mol_type = genomic RNA
cttcctgaccggcgcgcgcagcctgctgccgcggtcagcgcctgctcctgctcctccgctcctcctgcgcggggtgctgaaacagcccggggaagtagagccgcctccggggagcccaaccagccgaacgccgccggcgtcagcagccttgcgcggccacagcatgaccgctcgcggcctggcccttggcctcctcctgctgctactgtgtccagcgcaggtgttttcacagtcctgtgtttggtatggagagtgtggaattgcatatggggacaagaggtacaattgcgaatattctggcccaccaaaaccattgccaaaggatggatatgacttagtgcaggaactctgtccaggattcttctttggcaatgtcagtctctgttgtgatgttcggcagcttcagacactaaaagacaacctgcagctgcctctacagtttctgtccagatgtccatcctgtttttataacctactgaacctgttttgtgagctgacatgtagccctcgacagagtcagtttttgaatgttacagctactgaagattatgttgatcctgttacaaaccagacgaaaacaaatgtgaaagagttacaatactacgtcggacagagttttgccaatgcaatgtacaatgcctgccgggatgtggaggccccctcaagtaatgacaaggccctgggactcctgtgtgggaaggacgctgacgcctgtaatgccaccaactggattgaatacatgttcaataaggacaatggacaggcaccttttaccatcactcctgtgttttcagattttccagtccatgggatggagcccatgaacaatgccaccaaaggctgtgacgagtctgtggatgaggtcacagcaccatgtagctgccaagactgctctattgtctgtggccccaagccccagcccccacctcctcctgctccctggacgatccttggcttggacgccatgtatgtcatcatgtggatcacctacatggcgtttttgcttgtgttttttggagcattttttgcagtgtggtgctacagaaaacggtattttgtctccgagtacactcccatcgatagcaatatagctttttctgttaatgcaagtgacaaaggagaggcgtcctgctgtgaccctgtcagcgcagcatttgagggctgcttgaggcggctgttcacacgctgggggtctttctgcgtccgaaaccctggctgtgtcattttcttctcgctggtcttcattactgcgtgttcgtcaggcctggtgtttgtccgggtcacaaccaatccagttgacctctggtcagcccccagcagccaggctcgcctggaaaaagagtactttgaccagcactttgggcctttcttccggacggagcagctcatcatccgggcccctctcactgacaaacacatttaccagccatacccttcgggagctgatgtaccctttggacctccgcttgacatacagatactgcaccaggttcttgacttacaaatagccatcgaaaacattactgcctcttatgacaatgagactgtgacacttcaagacatctgcttggcccctctttcaccgtataacacgaactgcaccattttgagtgtgttaaattacttccagaacagccattccgtgctggaccacaagaaaggggacgacttctttgtgtatgccgattaccacacgcactttctgtactgcgtacgggctcctgcctctctgaatgatacaagtttgctccatgacccttgtctgggtacgtttggtggaccagtgttcccgtggcttgtgttgggaggctatgatgatcaaaactacaataacgccactgcccttgtgattaccttccctgtcaataattactataatgatacagagaagctccagagggcccaggcctgggaaaaagagtttattaattttgtgaaaaactacaagaatcccaatctgaccatttccttcactgctgaacgaagtattgaagatgaactaaatcgtgaaagtgacagtgatgtcttcaccgttgtaattagctatgccatcatgtttctatatatttccctagccttggggcacatgaaaagctgtcgcaggcttctggtggattcgaaggtctcactaggcatcgcgggcatcttgatcgtgctgagctcggtggcttgctccttgggtgtcttcagctacattgggttgcccttgaccctcattgtgattgaagtcatcccgttcctggtgctggctgttggagtggacaacatcttcattctggtgcaggcctaccagagagatgaacgtcttcaaggggaaaccctggatcagcagctgggcagggtcctaggagaagtggctcccagtatgttcctgtcatccttttctgagactgtagcatttttcttaggagcattgtccgtgatgccagccgtgcacaccttctctctctttgcgggattggcagtcttcattgactttcttctgcagattacctgtttcgtgagtctcttggggttagacattaaacgtcaagagaaaaatcggctagacatcttttgctgtgtcagaggtgctgaagatggaacaagcgtccaggcctcagagagctgtttgtttcgcttcttcaaaaactcctattctccacttctgctaaaggactggatgagaccaattgtgatagcaatatttgtgggtgttctgtcattcagcatcgcagtcctgaacaaagtagatattggattggatcagtctctttcgatgccagatgactcctacatggtggattatttcaaatccatcagtcagtacctgcatgcgggtccgcctgtgtactttgtcctggaggaagggcacgactacacttcttccaaggggcagaacatggtgtgcggcggcatgggctgcaacaatgattccctggtgcagcagatatttaacgcggcgcagctggacaactatacccgaataggcttcgccccctcgtcctggatcgacgattatttcgactgggtgaagccacagtcgtcttgctgtcgagtggacaatatcactgaccagttctgcaatgcttcagtggttgaccctgcctgcgttcgctgcaggcctctgactccggaaggcaaacagaggcctcaggggggagacttcatgagattcctgcccatgttcctttcggataaccctaaccccaagtgtggcaaagggggacatgctgcctatagttctgcagttaacatcctccttggccatggcaccagggtcggagccacgtacttcatgacctaccacaccgtgctgcagacctctgctgactttattgacgctctgaagaaagcccgacttatagccagtaatgtcaccgaaaccatgggcattaacggcagtgcctaccgagtatttccttacagtgtgttttatgtcttctacgaacagtacctgaccatcattgacgacactatcttcaacctcggtgtgtccctgggcgcgatatttctggtgaccatggtcctcctgggctgtgagctctggtctgcagtcatcatgtgtgccaccatcgccatggtcttggtcaacatgtttggagttatgtggctctggggcatcagtctgaacgctgtatccttggtcaacctggtgatgagctgtggcatctccgtggagttctgcagccacataaccagagcgttcacggtgagcatgaaaggcagccgcgtggagcgcgcggaagaggcacttgcccacatgggcagctccgtgttcagtggaatcacacttacaaaatttggagggattgtggtgttggcttttgccaaatctcaaattttccagatattctacttcaggatgtatttggccatggtcttactgggagccactcacggattaatatttctccctgtcttactcagttacatagggccatcagtaaataaagccaaaagttgtgccactgaagagcgatacaaaggaacagagcgcgaacggcttctaaatttctagccctctcgcagggcatcctgactgaactgtgtctaagggtcggtcggtttaccactggacgggtgctgcatcggcaaggccaagttgaacaccggatggtgccaaccatcggttgtttggcagcagctttgaacgtagcgcctgtgaactcaggaatgcacagttgacttgggaagcagtattactagatctggaggcaaccacaggacactaaacttctcccagcctcttcaggaaagaaacctcattctttggcaagcaggaggtgacactagatggctgtgaatgtgatccgctcactgacactctgtaaaggccaatcaatgcactgtctgtctctccttttaggagtaagccatcccacaagttctataccatatttttagtgacagttgaggttgtagatacactttataacattttatagtttaaagagctttattaatgcaataaattaactttgtacacatttttatataaaaaaacagcaagtgatttcagaatgttgtaggcctcattagagcttggtctccaaaaatctgtttgaaaaaagcaacatgttcttcacagtgttcccctagaaaggaagagatttaattgccagttagatgtggcatgaaatgagggacaaagaaagcatctcgtaggtgtgtctactgggttttaacttatttttctttaataaaatacattgttttcctaagttttggggttaccctatctgctttgagagacaaatacaaaagctaaatggaagaga

SEQ ID NO: 3 - protein, 151 aa, Homo sapiens
MRFLAATFLLLALSTAAQAEPVQFKDCGSVDGVIKEVNVSPCPTQPCQLSKGQSYSVNVTFTSNIQSKSSKAVVHGILMGVPVPFPIPEPDGCKSGINCPIQKDKTYSYLNKLPVKSEYPSIKLVVEWQLQDDKNQSLFCWEIPVQIVSHL

SEQ ID NO: 4 - RNA, 821 nt, Homo sapiens
  source 1..821: mol_type = genomic RNA
ctttcccgagcttggaacttcgttatccgcgatgcgtttcctggcagctacattcctgctcctggcgctcagcaccgctgcccaggccgaaccggtgcagttcaaggactgcggttctgtggatggagttataaaggaagtgaatgtgagcccatgccccacccaaccctgccagctgagcaaaggacagtcttacagcgtcaatgtcaccttcaccagcaatattcagtctaaaagcagcaaggccgtggtgcatggcatcctgatgggcgtcccagttccctttcccattcctgagcctgatggttgtaagagtggaattaactgccctatccaaaaagacaagacctatagctacctgaataaactaccagtgaaaagcgaatatccctctataaaactggtggtggagtggcaacttcaggatgacaaaaaccaaagtctcttctgctgggaaatcccagtacagatcgtttctcatctctaagtgcctcattgagttcggtgcatctggccaatgagtctgctgagactcttgacagcacctccagctctgctgcttcaacaacagtgacttgctctccaatggtatccagtgattcgttgaagaggaggtgctctgtagcagaaactgagctccgggtggctggttctcagtggttgtctcatgtctctttttctgtcttaggtggtttcattaaatgcagcacttggttagcagatgtttaattttttttttaacaacattaacttgtggcctctttctacacctggaaatttactcttgaataaataaaaactcgtttgtcttgtcttctgca